(19) United States
(12) Patent Application Publication
(10) Pub. No.: US 2005/0054877 A1
(43) Pub. Date: Mar. 10, 2005

(54) Enantiomerically selective cyclopropanation

(76) Inventor: Dennis Taylor, Burnsides (AU)

Correspondence Address: John E Vandigriff, Suite 200, 190 N Stemmons Frwy, Lewisville, TX, 75067, US

(21) Appl. No.: 10/474,149

(22) PCT Filed: Apr. 2, 2002
(86) PCT No.: PCT/AU02/00417

(30) Foreign Application Priority Data
Apr. 2, 2001 (AU) PR 4134

(51) Int. Cl. C07C 51/31

(52) U.S. Cl. 562/401

(57) ABSTRACT

A method of forming a cyclopropane having enhanced chirality said method comprising reacting together: 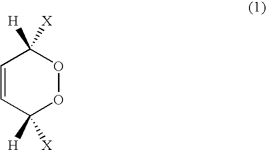 a symmetrical 1,2-dioxine of the formula (1), wherein X and Y are the same and are groups in which a carbon atom is bonded to the dioxine backbone; and a phosphorus ylide or a phosphorus ylide precursor, in the presence of a cobalt catalyst containing a chiral ligand.

### FIELD OF THE INVENTION

The present invention relates to a process for introducing an asymmetric cyclopropyl group into a molecule

### BACKGROUND OF THE INVENTION

Cyclopropanes containing natural and non-natural products are receiving considerable attention as synthetic targets since the incorporation of the rigidified cyclopropyl skeleton into bioactive analogues leads to conformationally constrained molecules. Such modifications often have significant effects on bioactivities with concomitant medical implications. Cyclopropanes are also generated as transient species in primary and secondary metabolisms in man, plants and microorganisms. Thus, the great importance of functionalised cyclopropanes in organic synthesis spurs a continuing search for efficient stereo controlled cyclopropanation methodologies. Of particular importance is the deficiency in methods for the construction of diversely functionalised cyclopropanes that contain greater than di-substitution. Chemicals containing the cyclopropyl moiety may be used inter alia: in the preparation of cyclopropyl amino acids; the preparation of cyclopropyl pharmaceuticals; the preparation of cyclopropyl pesticides; the preparation of new cyclopropyl products for furrier biological evaluation by chemistry and biochemistry laboratories as well as the preparation of isotopically labelled cyclopropanes as bioactive probes.

Cyclopropanes may be synthesised by a number of techniques including (i) the direct carbene transfer (both stoichiometric and catalytic) from a diazo precursor to an olefin utilizing transition metals (Rh, Cu, Zn, and Pd) and (ii) Michael addition of nucleophiles (usually sulphur ylides) to α, β-unsaturated ketones and esters followed by intra-molecular cyclization.

Such methods, employing as they do, potentially explosive material or requiring reaction mixtures that are highly sensitive to moisture have certain practical drawbacks.

An alternative reaction scheme for the synthesis of cyclopropanes involves the reaction between 1,2-dioxines and stabilised phosphorus ylides, is illustrated in reaction scheme 1.

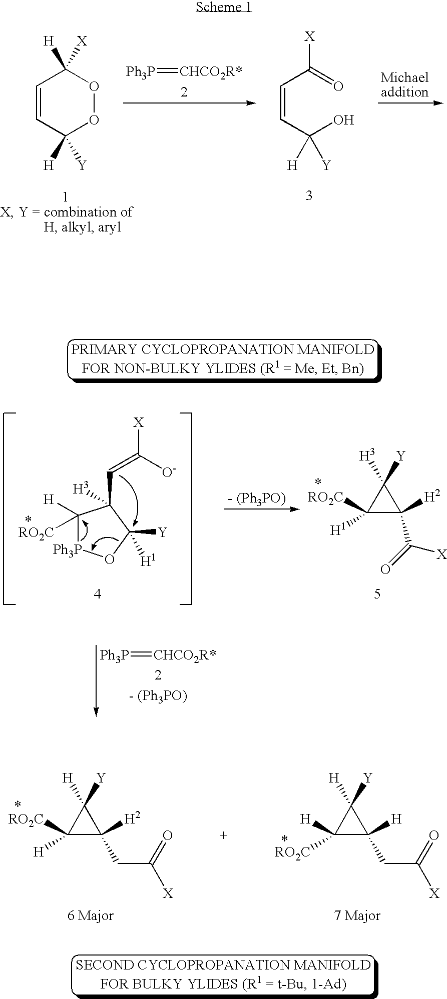

It is suggested that the ylide acts as a mild base inducing the ring opening of the 1,2-dioxine (1) to their isomeric cis γ-hydroxy enones (3) followed by Michael addition of the y]ide to the enone and attachment of the electrophilic phosphorus pole of the ylide to the hydroxyl moiety affording the intermediate 1-2λ-oxaphospholanes (4). Cyclization of the resultant enolate produces th cyclopropanes (5) in excellent yield. The cyclopropane products emerging from this reaction scheme have in the past been prepared as racemic mixtures.

The present invention is directed to a synthetic route for the preparation of optically enriched cyclopropane compounds.

### SUMMARY OF THE INVENTION

Therefore according to a first aspect of the invention there is provided a method of forming a cyclopropane having enhanced chirality said method comprising reacting together: 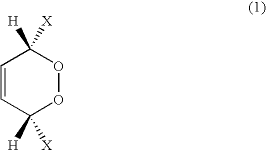
(i) a symmetrical 1,2-dioxine of the formula (1), wherein X and Y are the same and are groups in which a carbon atom is bonded to the dioxine backbone; and
(ii) a phosphorus ylide or a phosphorus ylide precursor; in the presence of
(iii) a cobalt catalyst containing a chiral ligand.

Preferably, the cobalt catalyst hils one of the structures shown below 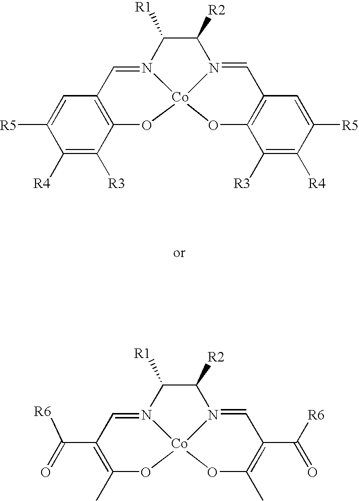
wherein R1 and R2 may be hydrogen, alkyl, aryl; R3 may be hydrogen, alkyl, t-alkyl, alkyl or aryl, alkoxy, aryl alkoxy; R3, R4, R5 and R6 may be independently H, alkyl, aryl, keto, or ester

In accordance with a further aspect of the invention there is provided a chiral cyclopropanation catalyst of the formulae shown below in Table 1 based on Cobalt Salen compounds and on Cobalt Beta-Ketoiminato compounds.

### Chiral Cobalt Salen Based Catalysts

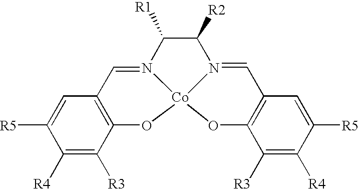 [0016] 8a: R1,R2=—(CH₂)₄—, R3=t-Bu, R4=H, R5=t-Bu (R,R isomer)
8b: R1,R2=—(CH₂)₄—, R3=R4=R5=H (R,R isomer)
8c: R1,R2=—(CH₂)₄—, R3=t-Bu, R4=R5=H (R,R isomer)
8d: R1,R2=Ph, R3=R4=R5=H (R,R isomer)
8e: R1,R2=Ph, R3=t-Bu, R4=R5=H (R,R isomer)
8f: R1,R2=—(CH₂)₄—, R3=S-PhEtCH—,R4=R5=H (R,R isomer)
8g: R1,R2=Ph, R3=S-PhEtCH—, 14=R5=H (R,R isomer)
8h: R1,R2=Ph, R3=S-PhEtCH—, 14=R5H (S,S isomer)

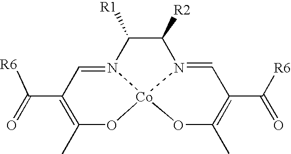

### Beta-Ketoiminato Cobalt Based Catalysts

9a: R1=R2=H, R6=(−)-bornoxy
9b: R1=R2=Ph, R6=(−)-bornoxy (S,S isomer)
9c: R1=R2=Ph, R6=(−)-bornoxy (R,R isomer)
9d: R1=R2=—(CH₂)₄—, R6=(−)-bornoxy(R,R isomer)
9e: R1=R2=Ph, R6=(−)-menthoxy (S,S isomer)
9f: R1=R2=Ph, R6=(−)-menthoxy (R,R isomer)
9g: R1=R2=Ph, R6=(+)-menthoxy (S,S isomer)
9h: R1=R2=Ph, R6=(+)-menthoxy (R,R isomer)
9i: R1=R2=Ph, R6=ethoxy (S,S isomer)
9j: R1=R2=Ph, ethoxy (R,R isomer)
9k: R1=R2=—(CH₂)₄—, R6=ethoxy (R,R isomer)
9l: R1=R2=Ph, R6=methyl (S,S isomer)
   Table 1

Catalysts according to the invention in the forms 8a to 8h may be prepared by reaction of Cobalt acetoacetate with the relevant SALEN ligand. SALEN ligands may be prepared by reaction of (1S, 2S) or (1R, 2R)-1,2-diphenylethylenediamine and the corresponding salicyaldehydes in ethanol under nitrogen atmosphere.

The catalysts 9a-9l may be prepared from reaction of (S,S) or (R,R)-1,2-diphenyl-ethylenediamine and the relevant β-diketone derivative, followed by subsequent reaction with Co acetate. Corresponding ligands can be made from the (1S, 2S) and (1R, 2R) cyclohexyl-1,2-diamine. It can be seen that each of the SALEN and the beta-ketoiminato based catalysts has a common structure around the cobalt atom, namely,
—(OR′C═R′CR′C═NR′CHCHR′N═CR′CR′═COR′)—.

The catalysts each include at least one chiral grouping. It is possible for more than one chiral grouping to exist in the catalyst and the values of R′ are wide. Each of the R′ groups may independently be H, alkyl, aryl, keto, ester and a range of other functionalities.

In the context of the present invention a phosphorus ylide or a phosphorus ylide precursor may be taken to mean a stabilized phosphorus ylide or a reactive compound capable of forming a phosphorus ylide in site.

In a still further aspect of the present invention there is provided a method of forming a cyclopropane having enhanced chirality as described above in which the phosphorus ylide or phosphorus ylide precursor is a compound of the formula:
(i) R7R8R9P═CZCO₂R10; wherein R7, R8 and R9 may be the same or different and may be alkyl, substituted alkyl, aryl, substituted aryl; R10 represents a non bulky group such as C₁₋₄ alkyl or substituted alkyl, or a bulky group such as 1-adamantyl; and Z represents hydrogen or methyl; or
(ii) R7R8R9P═CONR10R11; wherein R7, R8, R9 and R10 have the values identified in (i) above and wherein R11 represents a C₁₋₁₂ alkoxy grouping; or
(iii) R7R8R9P═CN; or
(iv) (R7O)₂P(O)CH₂CO₂ R10 wherein R7 and R10 have the values identified in (i) above.

Phosphorus ylides or a phosphorus ylide precursors of type (i) are exemplified by compound (2); type (ii) by compound (10); type (iii) by compound (11); and type (iv) by compound (12).

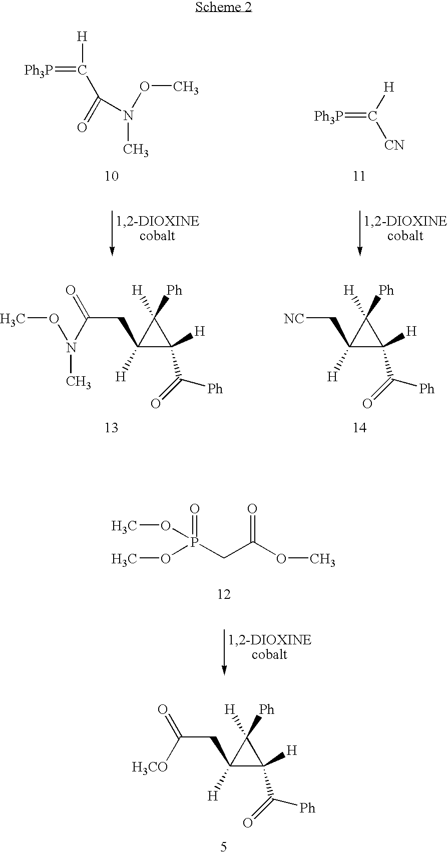

Preferably, the phosphorus ester ylide is a non-bulky stabilized ylide such as a benzyl 2-(triphenyl-λ-phosphanylidene) acetate (hereinafter referred to as a benzyl ester ylide), or, alternatively, a bulky ylide such as t-butyl 2-(triphenyl-λ-phosphanylidene) acetate (hereinafter referred to as a t-butyl ester ylide). Alternative phosphorus ylides or ylide precursors include N,N-methoxymethyl-2-(1,1-triphenyl-λ⁵-phosphanylidene)acetamide; 2-(1,1,1-triphenyl-λ⁵-phosphanylidene) acetonitrile or methyl 2-(dimethoxyphosphoryl)acetate. Examples of the reaction between the phosphorus ylides and phosphorus ylide precursors with 1,2-dioxines and a chiral cobalt catalyst and the resulting chiral cyclopropanes are shown below:

Preferably, the cyclopropanes of the invention are prepared in a solvent such as acetonitrile, ethyl acetate/hexane admixtures, ethyl acetate, tetrahydrofuran, ether, toluene, acetone, carbon tetrachloride, and dichloromethane or mixtures thereof. In a particularly preferred form of the invention the cyclopropanes of the invention are prepared in tetrahydrofuran. It will be appreciated by those skilled in the art that a range of solvents would in fact be suitable for wise in conducting the reaction of the invention. In any specific case an optimum solvent can be identified by trial and experiment using the above solvents and others. However, it should also be noted that alcohol is an unsuitable solvent for the reaction.

The enantiomerically enhanced cyclopropanes of the invention are preferably prepared with catalyst concentration of up to 50 mol % catalyst relative to the 1,2-dioxine, and more preferably still a catalyst concentration of 1-15 mol %.

The optically enriched cyclopropanes prepared in accordance with the invention may be further enriched by conventional techniques. For example, by recrystallization or conversion to diastereoisomers which can be separated by conventional techniques, e.g. recrystallization or column chromatography.

### DESCRIPTION OF THE PREFERRED EMBODIMENTS

The present invention will now be described by way of a number of non limiting examples. It should be noted that the invention presents a synthetic route for optically enhanced cyclopropanes and that the range of structures capable of synthesis according to the process of the invention is not to be take as being limited to those structures described herein. An extremely wide range of cyclopropane structures may be manufactured according to the process of the invention.

1. Catalyst Preparation

Beta Ketoiminato Catalyst Preparation—The preparation of a chiral cobalt catalyst (9b)

(a) Preparation of (−)-Bornyl Acetoacetate

To a solution of ethyl acetoacetate (4.5 g, 0.0346 mol) in n-heptane (70 mL) was added (−)-borneol (5.34 g, 0.0346 mol) and sodium hydride (50 mg) and the reaction mixture refluxed under Deans Stark conditions for 2 days. The solvent was then removed in vacuo and the residue purified by chromatography on silica (1:9 ethyl acetate:hexanes). (6.16 g, 70%) R_{f} 0.32 (1:9 ethyl acetate:hexanes) ¹H NMR δ 0.85 (s, 3H), 0.88 (s, 3H), 0.91 (s, 3H), 0.94-1.43 (m, 4H) 1.64-2.00 (m, 3H) 2.85 (s, 3H), 3.47 (s, 2H), 4.95 (ddd, 1H, J=15, 5.1, 3.3 Hz). ¹³C NMR δ 13.35, 18.71, 19.57, 26.96, 27.89, 29.99, 36.51, 44.76, 47.77, 48.76, 50.29, 81.07, 90.10, 167.30. MS m/z 238 (42%, M⁺), 154 (11%), 137 (100%), 121 (10%), 95 (23%).

(b)(−) Bornyl 2-Formyl-3-Oxobutanoate

To the (−)-bornyl acetoacetate (2.0 g, 8.4×10⁻³ moles) was added N,N-dimethylformamide dimethyl acetal (2 g, 1.68×10⁻² moles), the mixture stirred at room temperature for 2 hours, then cooled to 0° C. and methanolic sodium hydroxide (1N, 14 mL 1:l methanol:water) added. The reaction mixture was stirred for a further 2 hours then cooled to 0° C. and hydrochloric acid (1N) added till pH 3-4. The mixture was extracted with diethyl ether, dried (Na₂SO₄) and the solvent removed in vacuo. R_{f} 0.6 (1:9 ethyl acetate:hexanes) Due to the instability of the aldehyde it was used directly in the next reaction to form the ligand. ¹H NMR δ 0.875 (s, 3H), 0.90 (s, 3H), 0.94 (s, 3H), 1.00-1.48 (m, 1H), 1.12-1.48 (m, 4H), 2.32-2.54 (m, 1H), 2.57 (s, 3H), 4.96-5.05 (m, 1H), 9.25 (d, 1H, J-9 Hz).

The crude (−)-bornyl 2-formyl-3-oxobutanoate (0.25 g, 9.49×10⁻⁴ mol) was combined in ethanol (3 mL) with (1S, 2S)-1,2-diphenylethylenediamine (0.10 g, 4.75×10⁻⁴ mol) and allowed to react for 2 days, the solvent was then removed in vacuo and the residue purified by chromatography (1:9 acetone:dichloromethane) (290 mg, 86%). R_{f} 0.64 (1:9 acetone:dichloromethane). ¹H NMR δ0.79 (s, 6H), 0.86 (s, 6H), 0.90 (s, 6H), 0.96-1.32 (m, 4H), 1.60-1.82 (m, 6H), 2.28-2.49 (m, 2H), 2.51 (s, 6H), 4.67 (d, 2H, J=7.8 Hz), 4.89 (dm, 2H, J=8.2 Hz), 7.10-7.15 (m, 4H), 7.27-7.33 (m, 6H), 7.79 (d, 2H, J=12.8 Hz), 11.93-12.02 (m, 2H). ¹³C NMR δ 13.63, 18.84, 19.70, 27.64, 28.03, 31.02, 36.92, 44.84, 47.74, 48.74, 69.53, 79.48, 102.05, 135.68, 158.96, 167.09, 199.71. MS m/z 710 (M⁺, 4%), 555 (22%), 147 (19%), 354 (14%), 200 (31%), 137 (33%), 95 (100%).

(c) Preparation of Catalyst 9b)

A mixture of ligand (144 mg, 2.03×10⁻⁴ mol) was combined with cobalt acetate tetrahydrate (50.6 mg, 2.03×10⁻⁴) in deaerated ethanol (4 mL) and heated under reflux for 4 hours. The solvent was then removed in vacuo till dryness of the complex resulted. The complex was used without further purification.

Each of the catalysts 9a-9l may be prepared using similar methodology and by substitution of the appropriate bornoxy, ethoxy, methoxy, ethoxy or methyl starting materials.

2. Cyclopropane Preparation Using Chiral Beta Ketoiminato Based Catalyst (9a-9l)

(a) Preparation of Benzyl 2-(2-Benzoyl-3Phenylcyclopropyl) Acetate

3,6-Diphenyl-3,6dihydro-1,2 dioxine (1a) was reacted with the benzyl 2-(triphenyl-λ⁵-phosphanylidene)acetate (referred to as benzyl ester ylide) of in the presence of Cobalt (II) catalysts of type 9a-9l with as depicted in scheme 2 to produce the cyclopropane product.

The cobalt catalyst 9b (4.8 mg, 6.3×10⁻⁶ mol, 5 mol %) was dissolved in dichloromethane (1 mL) and allowed to equilibrate at the temperature of the reaction. 3,6-Diphenyl-3,6-dihydro-1,2-dioxine (20 mg, 8.4×10⁻⁵ mol) was added and the reaction left until such time as complete rearrangement of the dioxine had occurred

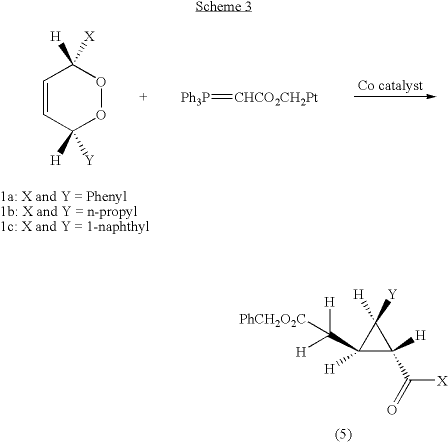

(t.l.c.). Benzyl ester ylide (40 mg, 9.8×10⁻⁵ mol) was then added and the reaction mixture left for 10 hours. The solvent was then removed and the residue purified by column chromatography (SiO₂, 3:17 ethyl acetate:hexanes).

It is to be noted that the scope of the, invention is not limited to the specific 1,2-dioxines listed in scheme 3. A much greater range of substrates may be used to form a wide variety of substituted cyclopropane products. It should however be noted that the 1,2-dioxine must be a symmetrical dioxine.

(b) Determination of Enantiomeric Excess

Utilising the cyclopropane synthesised in 2(a) above, (5 mg) was dissolved in 1:4 deuterated-benzene:carbon tetrachloride and enantiomeric excess determined by chiral shift n.m.r. techniques employing a europium tris[3-(heptafluoropropylhydroxy-methylene) -(+)-camphorate] complex. Table 2 illustrates the enantiomeric excess observed in the cyclopropane compound prepared in 2(a). In each case it can be seen that there is a significant enantiomeric excess indicating that the reaction product has enhanced chirality.

TABLE 2

| | Catalyst | Enantiomeric | Enantiomeric |
|---|---|---|---|
| Catalyst Number | Concentration | Ratio | Excess |
| 9a | 10 | 57/43 | 14 |
| 9b | 7.5 | 72/28 | 44 |
| 9c | 7.5 | 33/67 | 34 |
| 9d | 7.5 | 27/73 | 46 |
| 9e | 7.5 | 64/36 | 28 |
| 9f | 7.5 | 21/79 | 58 |
| 9g | 7.5 | 80/20 | 60 |
| 9h | 7.5 | 35/65 | 30 |
| 9i | 10 | 73/27 | 46 |
| 9j | 10 | 27/73 | 46 |
| 9k | 10 | 29/71 | 42 |
| 9l | 10 | 73/27 | 46 |

3 Comparison of Cyclopropane Preparation from Various 1,2-Dioxines

Cyclopropane compounds according to the invention were prepared from various 1,2-dioxine starting materials utilising catalysts 9b, 9i and 9l as identified above and the degree of chirality in the cyclopropane products compared by measurement of the enantiomeric excess as described above. The 3,6-disubstituted-3,6-dihydro-1,2-dioxines and the benzyl ester ylide were reacted in dichloromethane at 20-22° C. with 7.5-10 mol % catalyst. The following symmetrical 1,2-dioxines were used:
1a 3,6-Diphenyl-3,6-dihydro-1,2-dioxine (X═Y═Ph)
1b 3,6-Dipropyl-3,6-dihydro-1,2-dioxine (X═Y═n-propyl)
1c 3,6-Dinapthyl-3,6-dihydro-1,2-dioxine (X═Y═1-napthyl)

In each case the catalyst used results in a product having enhanced enantioselectivity for range or 1,2-dioxines, as illustrated in Table 3. A diverse range of aryl and alkyl substituents in the 1,2-dioxine would produce a similar effect.

TABLE 3

| | Catalyst | Catalyst | Enantiomeric | Enantiomeric |
|---|---|---|---|---|
| 1,2-Dioxine | Number | Conc. | Ratio | Excess |
| 1a | 9b | 7.5 | 72/28 | 44 |
| | 9i | 10 | 73/27 | 46 |
| | 9l | 10 | 73/27 | 46 |
| 1b | 9b | 10 | 67/33 | 34 |
| | 9i | 10 | 66/34 | 32 |
| | 9l | 10 | 65/35 | 30 |
| 1c | 9b | 10 | 72/28 | 44 |

4. Cyclopropane Preparation Using Chiral Salen Based Catalyst (8a-8e)

Cobalt (II) Salens of the type depicted in 8a-8e were used as catalyst in the reaction between 3,6-diphenyl-3,6-dihydro-1,2-dioxine (1a) in the presence of a benzyl ester ylide conducted in dichloromethane or tetrahydrofuran (THF) at 20-22° C. with 2-5% mol % catalyst. The enantiomeric excess of the resulting cyclopropane product was measured by the technique described hereinabove. Results are tabulated in table 4 and indicate that the catalysts 8a to 8h induce enantioselectivity into the cyclopropane product for a range of symmetrical 1,2-dioxines. It is also interesting to note that, in the case of catalyst 8h, the enantiomeric excess is increased when THF is used as a solvent compared with dichloromethane.

In the case of catalysts 8f, 8g and 8h it can be seen that these catalysts contain more than one chiral centre.

TABLE 4

| | | | Catalyst | | Enan- |
|---|---|---|---|---|---|
| Catalyst | | 1,2- | Concen- | Enantiomeric | tiomeric |
| Number | Solvent | dioxine | tration | Ratio | Excess |
| 8a | Dichloromethane | 1a | 2 | 35/65 | 30 |
| 8b | Dichloromethane | 1a | 2 | 31/69 | 38 |
| 8c | Dichloromethane | 1a | 2 | 31/69 | 38 |
| 8d | Dichloromethane | 1a | 2 | 38/62 | 24 |
| 8e | Dichloromethane | 1a | 2 | 36/64 | 28 |
| 8a | Dichloromethane | 1b | 2 | 40/60 | 20 |
| 8b | Dichloromethane | 1b | 2 | 35/65 | 30 |
| 8c | Dichloromethane | 1b | 2 | 36/64 | 28 |
| 8d | Dichloromethane | 1b | 2 | 41/59 | 18 |
| 8e | Dichloromethane | 1b | 2 | 41/59 | 18 |
| 8b | THF | 1a | 5 | 28/72 | 44 |
| 8c | THF | 1a | 5 | 34/65 | 32 |
| 8d | THF | 1a | 5 | 27/73 | 46 |
| 8e | THF | 1a | 5 | 36/64 | 28 |
| 8f | dichloromethane | 1a | 5 | 35/65 | 30 |
| 8g | dichloromethane | 1a | 5 | 33/67 | 34 |
| 8h | dichloromethane | 1a | 5 | 75/25 | 50 |
| 8h | THF | 1a | 5 | 82/18 | 64 |

5. Effect of Catalyst Concentration on the Enantiomeric Excess

(a) Effect of a Beta-Ketoiminato Catalyst

In the reaction between 3,6-diphenyl-3,6-dihydro-1,2-dioxine with benzyl ester ylide at 20° in dichloromethane the effect of various concentrations of the catalyst 9b on the enantiomeric excess was measured. 20 mg 1,2-dioxine was employed for 1 mL of solvent. Table 5a gives details of the measured enantiomeric ratio and enantiomeric excess.

Attention should be drawn to the fact that in the absence of the catalyst (catalyst mol %=0) the reaction product is a racemic mixture with no enantiomeric excess, thereby indicating the effect of the catalyst on the process. The catalyst is most effective in the range 7.5 mol %.

TABLE 5a

| CATALYST | | |
|---|---|---|
| (MOL %) | ENANTIOMERIC Ratio | ENANTIOMERIC EXCESS |
| 0 | 50/50 | 0 |
| 2.5 | 58/42 | 16 |
| 5 | 69/31 | 38 |
| 7.5 | 73/27 | 46 |
| 10 | 71/29 | 42 |
| 15 | 70/30 | 40 |
| 20 | 69/31 | 38 |
| 35 | 70/30 | 40 |
| 50 | 60/40 | 20 |

(b) Effect of a Beta-Ketoiminato Catalyst in THF or Dichloromethane Solvents

TABLE 5b

| | CAT- | ENANTIOMERIC | ENANTIOMERIC |
|---|---|---|---|
| Solvent | ALYST | Ratio | EXCESS |
| THF | 9b | 84/16 | 68 |
| dichloromethane | 9b | 72/28 | 44 |
| THF | 9c | 82/18 | 64 |
| dichloromethane | 9c | 67/33 | 34 |
| THF | 9d | 73/27 | 46 |
| dichloromethane | 9d | 73/27 | 46 |
| THF | 9h | 75/25 | 50 |
| dichloromethane | 9h | 65/35 | 30 |
| THF | 9g | 86/14 | 72 |
| dichloromethane | 9g | 80/20 | 60 |

The reaction between 3,6-diphenyl-3,6-dihydro-1,2-dioxine and benzyl ester ylide at 20° C. in dichloromethane or THF was catalysed with the catalysts identified as 9b, 9c, 9d, 9h and 9g. 20 mg of 1,2-dioxine was employed for each 20 mL of solvent. In each case the catalyst concentration was maintained at 7.5 mol %. Results are shown in table 5b

(c) Effect of Solvent on Catalyst Performance

Reaction of Cobalt (II) catalyst 9b with 3,6-diphenyl-3,6-dihydro-1,2-dioxine (1a) in the presence of the benzyl ester ylide at 20° C. in a specific solvent. 20 mg 1,2-dioxine employed for 1 mL of solvent. Employing 5.0 mol % catalyst. Results are shown in table 5c

TABLE 5c

| | ENANTIOMERIC | ENANTIOMERIC |
|---|---|---|
| Solvent | Ratio | EXCESS |
| acetonitrile | 76/24 | 52 |
| 25% ethyl acetate/hexane | 78/22 | 56 |
| Ethyl acetate | 77/23 | 54 |
| THF | 84/16 | 68 |
| ether | 67/33 | 34 |
| toluene | 67/33 | 34 |
| acetone | 76/24 | 52 |
| Carbon tetrachloride | 67/33 | 34 |
| dichloromethane | 68/32 | 36 |

It can be seen that that the cobalt catalyst 9b induces varying degrees of enantioselectivity in this reaction for a range of solvents under the conditions mentioned. The optimum enantiomeric excess is induced when the THF was employed as solvent.

(d) Temperature Effects

Reaction of Cobalt (II) catalyst with 3,6 diphenyl-3,6-dihydro-1,2-dioxine (1a) in the presence of the benzyl ester ylide at a specific temperate in a specific solvent. 20 mg 1,2-dioxine employed for 1 mL of solvent. Employing 5.0 mol % catalyst. Results are shown in table 5(d).

The comclusion may be drawn from the results shwn in Table 5d that changing the temperature has a dramatic effect on the degree of enantioselectivity in this reaction in certain solvents. Lowering the temperature increase the observed enantiomeric excess.

TABLE 5d

| | | ENANTIO- | |
|---|---|---|---|
| | Temperature | MERIC | ENANTIOMERIC |
| Solvent | (° C.) | Ratio | EXCESS |
| dichloromethane | 20 | 69/31 | 38 |
| dichloromethane | 0 | 74/26 | 48 |
| dichloromethane | −10 | 78/22 | 56 |
| dichloromethane | −15 | 81/19 | 62 |
| dichloromethane | −20 | 86/14 | 72 |
| dichloromethane | −40 | 80/20 | 60 |
| THF | 20 | 84/16 | 68 |
| THF | 0 | 87/13 | 74 |
| THF | −15 | 88/12 | 76 |
| THF | −40 | 86/14 | 72 |

### 5 Preparation of Optically Enriched Benzyl 2-[2-Benzoyl-3-Phenylcyclopropyl]acetate

(a) Preparation Using Benzyl Ester Ylide

(i) Procedure

A solution of 3,6-diphenyl-3,6-dihydro-1,2-dioxine (0.5 g) in THF (20 mL) and a solution of catalyst 9b (120 mg) in THF (5 mL) were equilibrated at −15° C. for 30 minutes after which time the two solutions were combined and the temperature maintained at−15° C. The rearrangement to the γ-hydroxyenone was monitored by t.l.c. and when complete the benzyl ester ylide (1.0 g) was added and the reaction mixture stirred overnight. The solvent was then removed in vacuo and the residue purified by chromatography (silica) (3:17 ethyl acetate:hexanes). The enantiomeric excess was determined to be 72% (an enantiomeric ratio of 86/14). The isolated yield of the benzyl 2-[2-benzoyl-3-phenylcyclopropyl]acetate product vias determined to be 78%.

(ii) Recrystallization

The enriched optical isomer of the benzyl 2-[2-benzoyl-3-phenylcyclopropyl]acetate prepared in step (a) can be further enriched by conventional processes used to isolate optical isomers. Recrystallization of the product from hexane:dichloromethane (10:1) at ambient temperatures resulted in a Product having an enantiomeric ratio of 88/12.

(b) Preparation Using a Tert-Butyl Ester Ylide

Reaction of Cobalt (II) catalyst 9b with 3,6diphenyl-3,6-dihydro-1,2-dioxine (1a) in the presence of the tert-butyl ester ylide (a bulky ylide) at a specific temperature in a specific solvent. 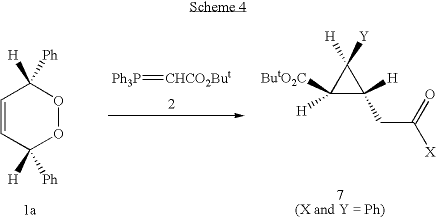
Experimental Procedure for the Above Reaction

A solution of catalyst 9b (12 mg, 0.5 mol %) in dichloromethane (0.1 mL) was added to an equilibrated solution of 3,6-diphenyl-3,6-dihydro-1,2-dioxine 1a (50 mg) in dichloromethane (2.9 mL) at 20° C. After rearrangement of the dioxine was complete lithium bromide (19 mg) was added, and allowed to stir for 5 minutes, followed by t-butyl ester ylide and the reaction mixture stirred vigorously for 5 days. The reaction mixture was filtered and the solvent was then removed in vacuo. Purification by chromatography (3:17 ethyl acetate:hexanes) gave the pure cyclopropane (40.1 mg, 57%) with an introduced enantiomeric excess of 69/31, 38%.

(b) Preparation of Cyclopropanes in D₂O

Cyclopropanes in accordance with the invention were prepared using catalysts of the invention as shown in scheme 5.

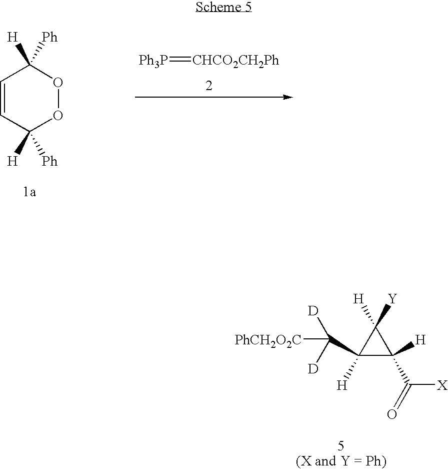

A solution of catalyst 9b (12 mg, 7.5 mol %) in dichloromethane (0.1 mL) was added to an equilibrated solution of 3,6-diphenyl-3,6-dihydro-1,2-dioxine (50 mg) in dichloromethane (2.9 mL) at 20° C. After rearrangement of the dioxine was complete deuterium oxide (1 mL) was added, and allowed to stir vigorously for 1 hour, followed by benzyl ester ylide and the reaction mixture stirred vigorously for 5 days. The deuterium oxide was removed employing a pipette off and the reaction mixture dried (Na₂SO₄). The solvent was removed in vacuo and the residue purified by chromatography (3:17 ethyl acetate: hexanes) to give the pure cyclopropane (49.9 mg, 67%) with an introduced enantiomeric excess of 75/25, 50%.

6. Comparison of Cyclopropane Preparation from Various Phosphorus Ylides and Precursors

Typical Procedure for the Preparation of Cyclopropane 5 Derived from the Combination of Phosphonate (12), Symmetrical 1,2-dioxine 1a and Cobalt Catalyst 9b.

To a solution of phosphonate 12 (45 mg) in THF (230 μl) at−78° C. was added methyl lithium (175 μl, 1.4 M) over 5 minutes and the reaction mixture stirred at−78° C. for 30 minutes and then allowed to attain ambient temperature. Separately the 1,2-dioxine 1a (60 mg) was combined with the cobalt catalyst 9b (12 mg) in THF (3 mL) and the dioxine allowed to rearrange. This, mixture was then cooled to −78° C. and the ylide generated above added drop wise via a syringe and the mixture stirred at −78° C. for 30 minutes before being allowed to attain ambient temperature. After 15 hours the solvent was removed in vacuo and the residue purified by column chromatography (3:17 ethyl acetate/hexanes) to afford the cyclopropane 5 in 79% yield. The ee was measured by chiral shift NM as described herein above.

The conditions used for the preparation of the cyclopropanes from the use of ylides of type 10 and 11 above are as described for the benzyl ester ylide.

Table 6: Enantiomeric excess observed in cyclopropanes of types 5, 13 and 14 depicted above made using symmetrical 1,2-dioxine 1a and ylides of type 10-12 (depicted above) in dichloromethane or tetrahydrofuran (THF) at 20-22° C. with 7.5 mol % of catalyst 9b. Note: the ylide of the phosphonate 12 is generated first with base, see procedure above.

| | | Catalyst | | Enantiomeric | Enantiomeric |
|---|---|---|---|---|---|
| Dioxine | Ylide | Number | Solvent | Ratio | Excess |
| 1a | 10 | 9b | dichloromethane | 71/29 | 42 |
| 1a | 11 | 9b | dichloromethane | 69/31 | 38 |
| 1a | 12 | 9b | THF | 83/17 | 66 |

The catalysts and processes of the invention therefore enable the synthesis of a range of isotopically labelled cyclopropane compounds, which are useful in a variety of situations.

7. Synthetic Schemes Using Optically Enriched Cyclopropanes

Scheme 6 illustrates an example of how an enantiomerically enhanced cyclopropane produced in the process of the present invention can be used in the synthesis of optically enriched amide products.

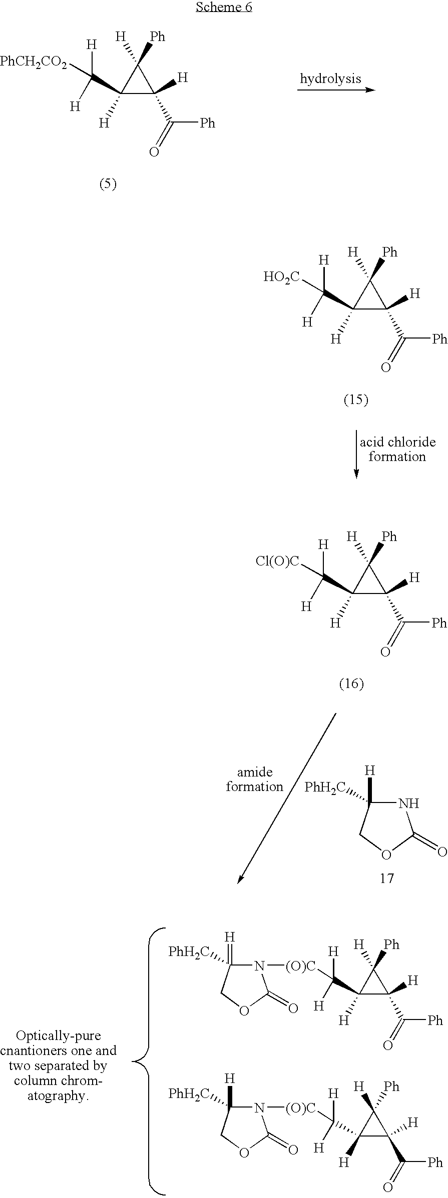

(a) Preparation of Trans (±) 2-[2-Benzoyl-3-Phenylcyclopropyl]Acetic Acid

Hydrolysis of cyclopropane 5 (ee ratio 86/14) prepared above afforded the acid (15). The acid product was found to have a similar ee to the cyclopropane starting material, that is, the material is optically enhances.

(b) Preparation of Trans 2-[2-Benzoyl-3-Phenylcycloproyl]Acetyl Chloride

To a solution of trans 2-[2-benzoyl-3-phenylcyclopropyl]acetic acid (15) (0.394 g) of ee cited above in dry dichloromethane 8 mL) and DMF (1 drop), at 0° C. under nitrogen, was added oxalyl chloride (0.291 g) in dichloromethane (4 mL) drop wise over 5 minutes. The reaction mixture was then stirred at 0° C. for 2 hours and a further 2 hours at room temperature. The volatiles were then removed to afford crude (16) which was used as is.

(c) Preparation of Optically Pure

(4S)3-{2-[1S, 2R, 3R)-2-Benzoyl-3-Phenylcyclopropyl]Acetyl}-4-benzyl-1,3-oxazolan-2-one and

(4S)3-{2-[(1R, 2S, 3S)-2-Benzoyl-3-Phenylcyclopropyl]Acetyl}-4-benzyl-1,3-oxazolan-2-one.

To a solution of (S)-4-benzyl-2-oxazolidinone (17) (0.252g) in THF (4 mL) with triphenylmethane (5 mg, indicator), at−78° C. under nitrogen, was added n-BuLi (1.9M in hexanes) until an orange colour persisted. The reaction mixture was then stirred at−78° C. for 1 hour after which time the acid chloride in THF (3 mL) was added via a cannula. The reaction was then stirred at −78° C. for 4 hours then poured into NB₄Cl (sat. 10 mL) immediately, diluted with water (10 mL) and extracted with dichloromethane (2×30 mL). The combined organic phases were dried (Na₂SO₄) and the solvent removed in vacuo. The residue was purified by chromatography (1:3, ethyl acetate:hexanes) with separation of the diastereomers.

The two enantiomers were characterised as follows:

First Enantiomer eluted (top spot) R_{f} 0.20 (1:5 ethyl acetate:hexanes). ¹H NMR (600 MHz) δ 2.42-2.46 (m, 1H), 2.67 (dd, 1H, J=13.2, 9.6 Hz), 2.93 (dd, 1H, J=17.4, 7.2 Hz), 2.99 (dd, 1H, J=17.4, 7.8 Hz), 3.16 (dd, 1H, J=9.6, 4.8 Hz), 3.23 (dd, 2H, J=9.6, 4.8 Hz), 4.06 (t, 1H, J=8.4 Hz), 4.08 (dd, 1H, J=9.3 Hz), 4.51-4.55 (m, 1H), 7.15 (d, 2H, J=7.2 Hz), 7.22-7.26 (m, 2H), 7.28-7.34 (m, 6H), 7.52 (t, 2H, J=7.8 Hz), 7.61 (tt, 1H, J=7.2, 1.2 Hz), 8.08-8.10 (m, 2H). ¹³C NMR δ 27.16, 29.01, 33.60, 33.83, 37.81, 55.08, 66.18, 126.93, 127.36, 128.19, 128.43, 128.69, 128.79, 128.94, 129.35, 133.05, 135.11, 135.88, 137.72, 171.61. IR (dichloromethane solution) 1668, 1703, 1780 cm⁻¹. MS m/z 440.2 (42%, [M+H]⁺), 422.2 (8%), 263.1 (100%), 221.1 (37% o). C₂₈H₂₅NO₄ requires C: 76.51, H: 5.74, found C: 76.63, H: 5.65. Accurate MS C₂₈H₂₅NO₄ requires 439.17836, found 440.18753 ([M+F]⁺); [α]²⁴=−39.24 (c=2.06, CH₂Cl₂)7

Second Enantiomer eluted (bottom spot) R_{f} 0.13 (1:5 ethyl acetate:hexanes). ¹H NMR (600 MHz) δ 2.46 (tt, 1H, J=7.8, 6.6 Hz), 2.59 (dd, 1H, J=13.8, 9 Hz), 2.93 (dd, 1H, J=18, 6.6 Hz), 2.99 (ddI, 1H, J=17.4, 7.8 Hz), 3.05 (dd, 1H, J=13.8, 3.6 Hz), 3.21 (d, 2H, J=6H), 4.09 (dd, 1H, J=9, 3 Hz), 4.13 (t, 1H, J=7.8 Hz), 4.60-4.64 (m, 1H), 7.02 (dd, 2H, J=7.8, 1.8 Hz), 7.24-7.28 (m, 4H), 7.33 (d, 4H, J=4.2 Hz), 7.51 (t, 2H, J=7.8 Hz), 7.59 (tt, 1H, J=7.8, 1.8 Hz), 8.09-8.10 (m, 2H). ¹³C NMR (600M) δ 26.94, 28.81, 33.75, 34.31, 37.47, 54.75, 66.09, 127.02, 127.34, 128.16, 128.48, 128.67, 128.88, 128.92, 129.35, 133.04, 134.87, 135.88, 137.72, 153.30, 171.45, 198.39. IR (dichloromethane solution) 1668, 1703, 1782 cm⁻¹. MS m/z 440.1 (45%, [M+H]⁺), 422.1 (10%), 263.1 (100%), 221.1 (34%). C₂₈H₂₅NO₄ requires C: 76.51, H: 5.74, found C: 76.49, H: 5.68. Accurate MS C₂₈H₂₅NO₄ requires 439.17836, found 440.18712 ([M+H]⁺).

The invention has been described by way of example. The examples are not, however, to be taken as limiting the scope of the invention in any way. Modifications and variations of the invention such as would be apparent to a skilled addressee are deemed to be within the scope of the invention.

1. A method of forming a cyclopropane having enhanced chirality said method comprising reacting together: 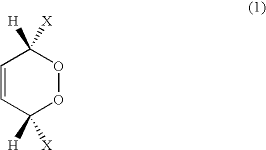
i) a symmetrical 1,2-dioxine of the formula (1), wherein X and Y are the same and are groups in which a carbon atom is bonded to the dioxine backbone; and
ii) a phosphorus ylide or a phosphorus ylide precursor; in the presence of
iii) a cobalt catalyst containing a chiral ligand.

2. A method of forming a cyclopropane having enhanced chirality according to claim 1, wherein the chiral ligand in the catalyst has the structure: —(OR′C═R′CR′C═NR′CHCHR′N═CR′CR′═COR′)— around the cobalt atom and wherein each R′ moiety may be independently selected from a wide range of groups including, but not limited to H, alkyl, aryl, keto, ester and a range of other functionalities.

3. A method of forming a cyclopropane having enhanced chirality according to claim 1, in which the cobalt catalyst has the structure: 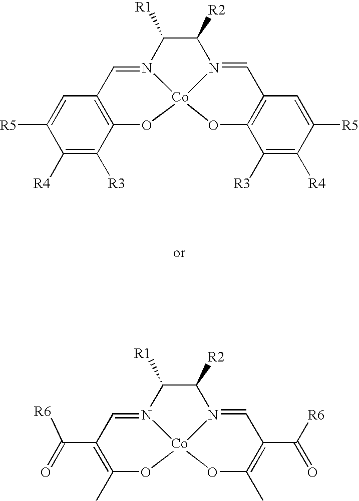
Wherein R1 and R2 may be hydrogen, alkyl, aryl; R3 may be hydrogen, alkyl, t-alkyl, alkyl or aryl, alkoxy, aryl alkoxy; R3, R4, R5 and R6 may be independently H, alky, aryl, keto, or ester.

4. A method of forming a cyclopropane having enhanced chirality according to claim 3, in which R1 and R2 are each the same and are selected from hydrogen, (CH₂)₄, or benzyl.

5. A method of forming a cyclopropane having enhanced chirality according to claim 3, in which R3 is hydrogen, t-butyl, or benzyl ethyl, bornoxy, menthoxy, ethoxy or methyl.

6. A method of forming a cyclopropane having enhanced chirality according to claim 3, in which R4 and R5 may be the same or different and are selected from hydrogen, and t-butyl.

7. A method of forming a cyclopropane having enhanced chirality according to claim 1 in which the phosphorus ylide or phosphorus ylide precursor may be taken to mean a stabilized phosphorus ylide or a reactive compound capable of forming a phosphorus ylide in situ.

8. A method of forming a cyclopropane having enhanced chirality according to claim 1, in which the phosphorus ylide or phosphorus ylide precursor is a compound of the formula:
i) R7R8R9P=CZCO₂R10; wherein R7, R8 and R9 may be the same or different and may be alkyl, substituted alkyl, aryl, substituted aryl; R10 represents a non bulky group such ac C₁₋₄ alkyl or substituted alky, or a bulky group such as 1-adamantyl; and Z represents hydrogen or methyl; or
ii) R7R8R9P═CONR10R11; wherein R7, R8, R9 and R10 have the values identified in (i) above and wherein R11 represents a C₁₋₁₂ alkoxy grouping;
iii) R7R8R9P═CN; or
iv) (R70)₂P(O)CH₂CO₂ R10 wherein R7 and R10 have the values identified in (i) above.

9. A method of forming a cyclopropane having enhanced chirality according to claim 1, in which the phosphorus ylide is a non-bulky stabilized ylide.

10. A method of forming a cyclopropane having enhanced chirality according to claim 9, in which the non-bulky stabilized ylide is benzyl 2-(triphenyl-λ-5 phosphanylidene) acetate.

11. A method of forming a cyclopropane having enhanced chirality according to claim 1, in which the phosphorus ylide is a bulky ylide.

12. A method of forming a cyclopropane having enhanced chirality according to claim 1, in which the bulky ylide is t-butyl 2-(triphenyl-λ-phosphanylidene) acetate.

13. A method of forming a cyclopropane having enhanced chirality according to claim 1, in which the phosphorus ylide or phosphorus ylide precursor is selected from N,N-methoxymethyl-2-(1,1,1-triphenyl-λ⁵-phosphanylidene)acetamide; 2-(1,1,1-triphenyl-λ⁵-phosphanylidene) acetonitrile or methyl 2-(dimethoxyphosphoryl) acetate.

14. A method of forming a cyclopropane having enhanced chirality according to claim 1, in which X and Y are selected from H, alkyl or aryl groups.

15. A method of forming a cyclopropane having enhanced chirality according to claim 1, wherein the reaction is conducted in a solvent selected from acetonitrile, ethyl acetate/hexane admixtures, ethyl acetate, tetrahydrofuran, ether, toluene, acetone, carbon tetrachloride, and dichloromethane or mixtures thereof.

16. A method of forming a cyclopropane having enhanced chirality according to claim 1, in which the reaction is conducted with catalyst concentration of up to 50 mol % catalyst relative to the 1,2-dioxine, and more preferably a catalyst concentration of 1-15 mol %.

17. A method of forming a cyclopropane having enhanced chirality according to claim 1, wherein the optically enriched cyclopropanes are subjected to recrystallization or column chromatography.

18. A method of forming a cyclopropane having enhanced chirality according to claim 1, in which the enhanced chirality of the product, as determined by chiral shift n.m.r. techniques and expressed as an enantiomeric ratio may be as high as 90/10.
